(19) United States
(12) Patent Application Publication
(10) Pub. No.: US 2003/0045821 A1
(43) Pub. Date: Mar. 6, 2003

(54) Device for treating an elevated concentration of interstitial fluid in a body area of a patient

(76) Inventor: Emily Iker, Santa Monica, CA (US)

Correspondence Address: ROTHWELL, FIGG, ERNST & MANBECK, P.C., 1425 K STREET, N.W., SUITE 800, WASHINGTON, DC, 20005, US

(21) Appl. No.: 09/944,155

(22) Filed: Sep. 4, 2001

(51) Int. Cl. A61F 5/00

(52) U.S. Cl. 602/20; 602/61

(57) ABSTRACT

An apparatus for treating an elevated concentration of interstitial fluid in a body area of a patient includes a pad constructed and arranged to be placed in pressure-applying, conforming relation to the body area. The pad includes inner and outer liners connected at marginal edges thereof to one another, and a foam pad having a multiplicity of resilient protrusions is encased between the inner and outer liners. A plurality of channels are formed in areas between the protrusions by securing the inner liner in close proximity to the outer liner along each channel. The channels and protrusions are constructed and arranged such that when the pad is placed in pressure-applying, conforming relation to the body area, localized pressure generated on the body area at each protrusion is greater than localized pressure generated on the body area along the channel. The pad can be rolled into a garment, such as a sleeve, a vest, or a brief, and worn by the patient over the afflicted body area, for example, a limb or the pelvic area. Effective fluid redistribution is achieved by the combination of pressure applied at the protrusions and low pressure areas between protrusions and along the channels, which provide pathways along which the excess interstitial fluid may flow out of the afflicted body area.

### FIELD OF THE INVENTION

The present invention is directed to a device in the form of a garment to be worn on a body area of a patient and which applies surface pressure to the body area so as to redistribute elevated concentrations of interstitial fluid. The pressure is applied at a plurality of discrete elevated pressure areas with channels of low pressure area provided to permit the redistribution of the fluid, rather than as a uniform blanket surface pressure to the body area.

### BACKGROUND OF THE INVENTION

The lymphatic system is organized like the blood system in that it includes a system of numerous tiny vessels connected to a network of larger vessels, and through which system and network a liquid medium containing solutes and particulates is transferred. A healthy lymphatic system continuously drains lymphatic fluid, consisting of a mixture of lymph, water, proteins and other matter, away from various interstitial areas of the body and back into the blood system. Lymph is the clear, liquid medium or solvent of the lymphatic system.

The lymph fluid is pumped through the lymphatic system and away from various body areas by both the action of adjacent muscle tissue and the contraction of the larger lymphatic vessels. Foreign matter is filtered out of the lymph fluid as the fluid passes through bundles of lymph nodes during its course through the lymphatic system. The lymph nodes also monitor the contents of the lymph fluid to determine if any appropriate immune reactions should be initiated by the host's immune system. The lymph is then transferred back into the blood system after this filtration.

Lymphedema is the accumulation of excessive lymph fluid and swelling of subcutaneous tissues due to the obstruction or destruction of lymph nodes. It has a variety of causes, including infection, accidents, radiation therapy, and other surgical procedures, and most often occurs in the arms or legs but can also affect the trunk and breasts of a patient.

A variety of methods and devices have been proposed to treat lymphedema and other afflictions resulting in increased concentrations of interstitial fluid. Such devices include a sleeve worn on the affected limb that is connected to sequential pump which works like a multiplicity of blood pressure cuffs, extending from the distal end of the limb to the proximal end of the limb, which contract and expand sequentially. When pressure is exerted by any one chamber or cuff, it applies a ‘blanket pressure’ (i.e. an annular pressure which completely circumscribes the limb or other body portion) to the skin directly underneath the area of that chamber. When the pressure upon that area is released, a similar blanket pressure is then applied to the portion of the limb covered by the next adjacent chamber, and so on up the limb. Such blanket pressures are applied sequentially from the distal to the proximal ends of the limb, with the intention of forcing the fluids up the limb and into the trunk of the body, where existing lymph nodes can process them.

Another prior mode of treatment includes a double walled sheath or stocking in which air pressure is introduced between the walls to squeeze the limb. It has been found that this and other similar systems, which rely on uniform blanket pressure application through the length of the afflicted limb or portions thereof, do not perform very well and in fact may interfere with the desired distal-to-proximal flow of lymphatic fluid.

Other approaches to treatment have included employment of a sheath that is separated into a number of longitudinally spaced inflatable air cells encircling the limb to be treated. These cells are successively inflated with uniform air pressure from the distal end to the proximal end of the sheath with the intent of promoting fluid flow in the desired direction. Such systems have been largely ineffectual, as they rely on air pressure being maintained at the same level or magnitude in any one, or all, of the pressurized cells, producing a blanket effect.

Reid, U.S. Pat. No. 5,916,183, the disclosure of which is hereby incorporated by reference, describes a sheet of convoluted plastic foam which is encased between inner and outer linings and rolled onto a body area in such a manner as to push the extending elements, or fingers, of the convoluted foam sheet against a swollen body part. The foam fingers create a grid pattern of high and low pressure areas when pressed against the patient's body area. Adjustable straps and matching D ring straps, which extend circumferentially around the sleeve and are spaced longitudinally along the sleeve, are sewn into the outer lining, by which means the convoluted plastic foam sheet is secured and pressed against and/or around a body part.

The use of a heavy nylon fabric in the outer lining of the device of Reid limits the patient's range of motion and wearing comfort. In addition, the direct attachment of the strapping and matching D ring straps to the heavy nylon fabric increases the weight of the sleeve and securement of the straps reduces the uniformity of pressure exerted by individual fingers across the total circumference of a limb or body part. The employment of heavy nylon fabric also limits the adjustability of a particular device to a small range of arm circumferences or arm sizes.

The discrete high pressure point created by the protruding fingers and the associated low pressure regions created between the fingers provides both pressure for redistributing interstitial fluid and pathways which facilitate somewhat the redistribution of the fluid.

Kellogg, U.S. Pat. No. 5,976,099, the disclosure of which is hereby incorporated by reference, describes an apparatus including a multiplicity of resilient particles organized into pockets separated by seams. The seams are stitched so that the pockets form a chevron configuration. The particles are made of compressible, high resiliency, low density, open cell plastic foam. The foam is ground up in order to produce a mixture of different sized and shaped particles. This design is intended to create a series of relatively large pockets of high pressure areas and an intersecting network of narrow, lower pressure channels at the seams. The network of channels in intended to allow the interstitial fluid to flow into to the lymphatic system and/or out of and away from the body area which is under compression from the pockets of resilient particles. A commercial version of the device described in the Kellogg '099 patent is sold under the trade name Tribute™.

Another device similar to that described in Kellogg '099 is commercially available under the trade name Jovi PAK. The Jovi PAK device contains a multiplicity of resilient particles organized into pockets separated by seams. The seams are stitched so that the pockets, instead of forming a chevron configuration, form longitudinal lines that follow the anatomical flow of the body from a distal end of a limb to a proximal end of a limb. The particles in the Jovi PAK device are made of similar foam as in the Kellogg '099 apparatus. The Jovi PAK device is rolled into a closed sleeve which must be pulled onto and off the patient's limb like a stocking. Such a garment can be difficult for the patient to put on and remove without assistance.

Although each of these devices provides some measure of fluid volume reduction and improved tissue consistency, it has been discovered that each of them suffers from some deficiencies that affect patient comfort and limit effectiveness. None of these devices provide ease of use and washability while at the same time minimizing weight. There continues to exist a need in the treatment of lymphedema for a lightweight, easy to use, and washable device that maximizes the fluid volume reduction and provides maximum improvement in tissue consistency of afflicted areas.

### SUMMARY OF THE INVENTION

The forgoing shortcomings are addressed by the present invention in which a device for treating an elevated concentration of interstitial fluid in a body area of a patient includes a pad constructed and arranged to be placed in pressure-applying, conforming relation to the body area. The pad includes inner and outer liners connected at marginal edges thereof to one another. A foam pad having a multiplicity of resilient protrusions is encased between the inner and outer liners. A plurality of channels are formed in areas between the protrusions by securing the inner liner in close proximity to the outer liner along each channel. The channels and protrusions are constructed and arranged such that when the pad is placed in pressure-applying, conforming relation to the body area, localized pressure generated on the body area at each protrusion is greater than localized pressure generated on the body area along the channel.

The pad can be rolled into a garment, such as a sleeve, a vest, or a brief, and worn by the patient over the afflicted body area, for example, a limb or the pelvic area. Effective fluid redistribution is achieved by the combination of pressure applied at the protrusions and low pressure areas between protrusions and along the channels, which provide pathways along which the excess interstitial fluid may flow out of the afflicted body area.

It has been discovered in patient trial comparisons with the Tribute™ and Jovi PAK devices described above that the device of the present invention provides better improvement, in comparable treatment time, in terms of tissue consistency and fluid volume reduction. Moreover the device of the present invention is lighter than presently available pressure garments and is more easily washable, because it includes liners made of washable, lightweight cotton fabric. In a preferred embodiment, a Velcro strip is provided which permits the pad to be easily unrolled and removed from the body area.

Other objects, features, and characteristics of the present invention, including the methods of operation and the function and interrelation of the elements of structure, will become more apparent upon consideration of the following description and the appended claims, with reference to the accompanying drawings, all of which form a part of this disclosure, wherein like reference numerals designate corresponding parts in the various figures.

### BRIEF DESCRIPTION OF THE DRAWINGS

FIG. 1 is a perspective view illustrating the device of the present invention configured as an arm sleeve;

FIG. 2 is a perspective view illustrating the device of the present invention configured as a leg sleeve;

FIG. 3 is an illustration of the device of the present invention configured as a brief;

FIG. 4 is a perspective view illustrating the device of the present invention configured as an arm sleeve and shown in an open laid-out orientation;

FIG. 5 is a partial cross-sectional perspective view of a corrugated foam pad employed in the present invention; and

FIG. 6 is a partial cross-sectional view along the lines VI-VI in FIG. 4.

### DETAILED DESCRIPTION OF THE PREFERRED EMBODIMENT

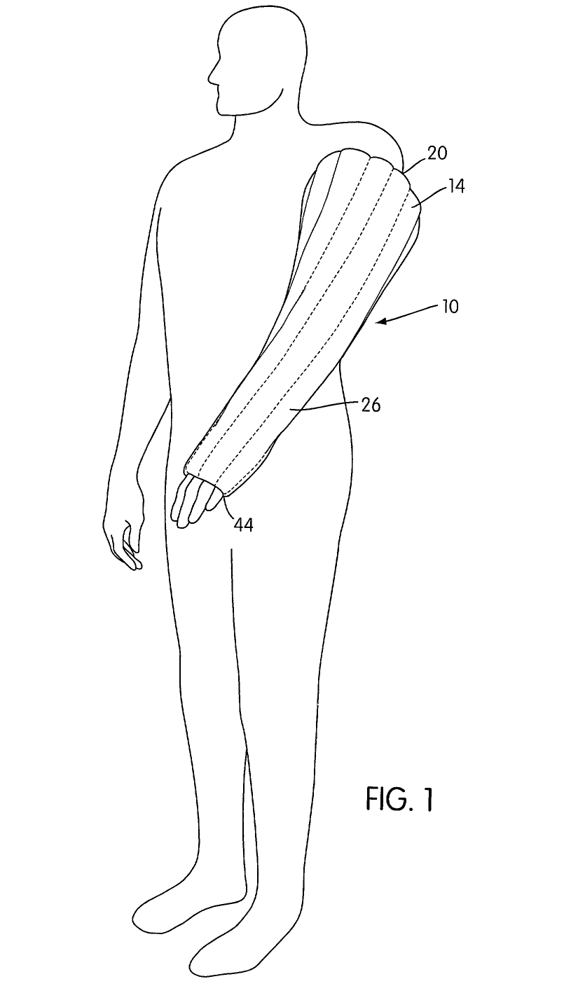

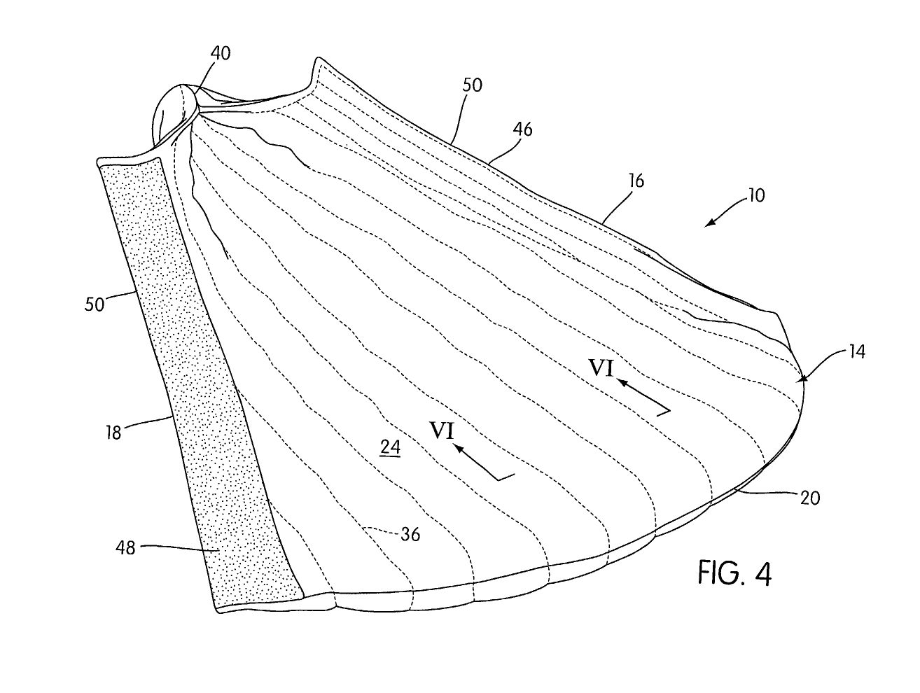

A detailed description of the preferred embodiment is shown in the figures. According to a preferred embodiment, the apparatus in the form of a garment, such as a sleeve 10 is shown in FIGS. 1 and 4. The sleeve 10 comprises a pad 14 with opposed edges 16, 18. The pad can be rolled so as to bring the opposed edges 16, 18 into proximity with each other so as to form a sleeve that can be worn over the limb (e.g., the arm or leg) of a patient. In a preferred embodiment, the pad is formed in such a manner as to form a tapered sleeve when in the rolled configuration. An upper edge 20 of the pad may be contoured for the comfort of the wearer while providing applied pressure as far up the treated limb as possible.

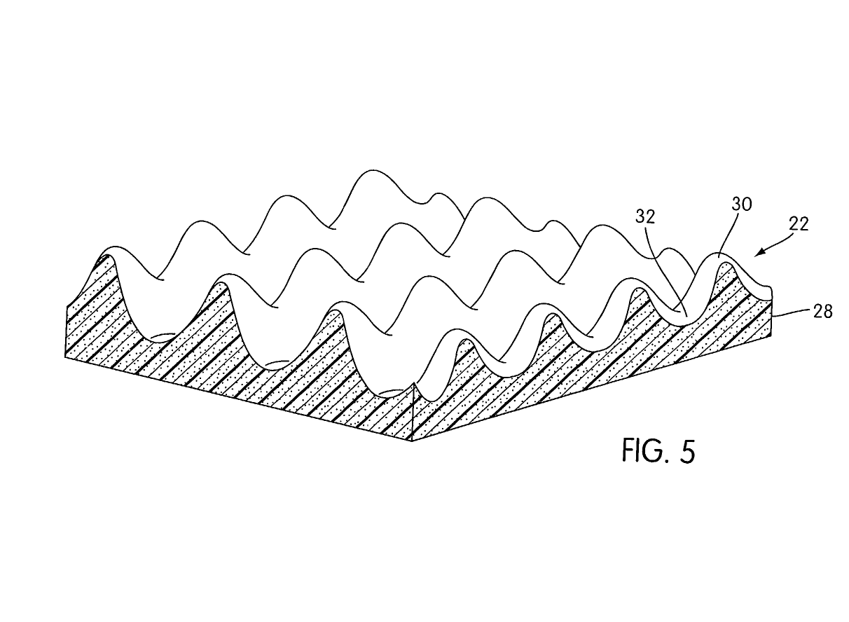

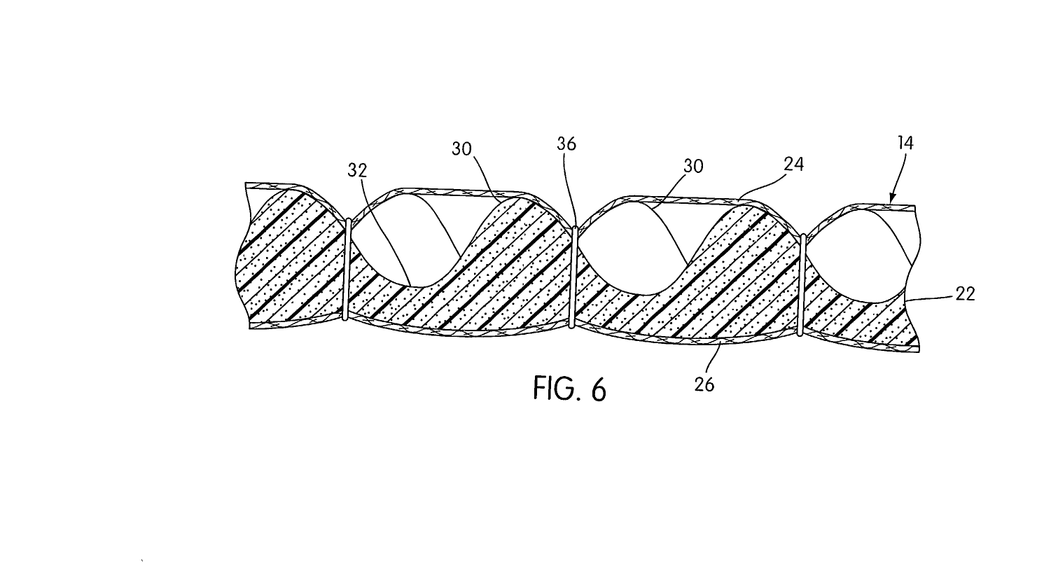

As shown in FIG. 6, the pad comprises a foam pad 22 encased in a liner formed by inner layer 24 and outer layer 26. The inner and outer layers 24, 26 are preferably a lightweight cotton fabric. The foam pad 22 is preferably of the form shown in FIG. 5 and includes a base area 28 and a plurality of upstanding protrusions 30 and intermediate valleys 32. Medical grade orthotic foam available from medical supply companies has proven effective.

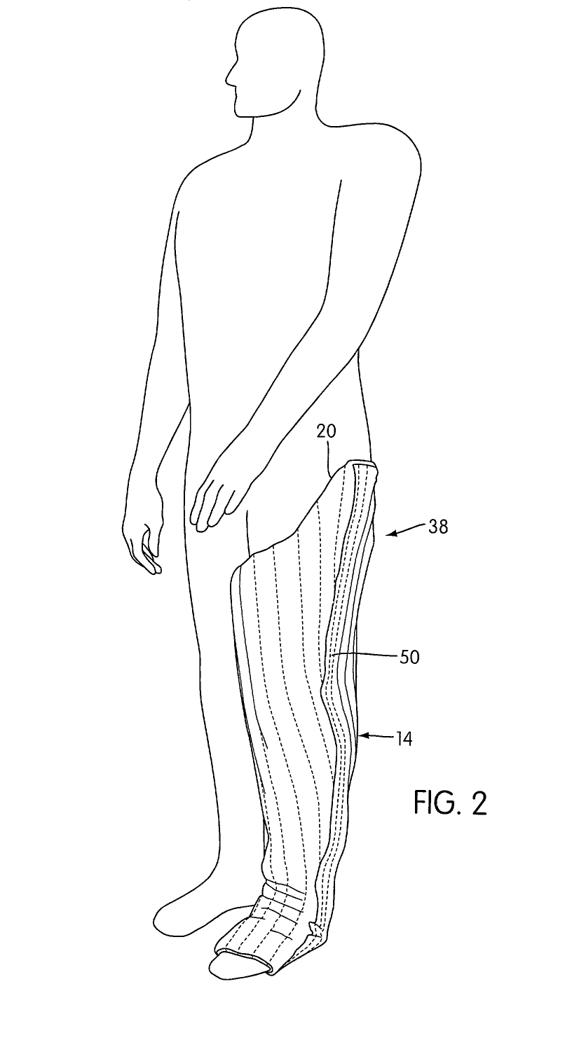

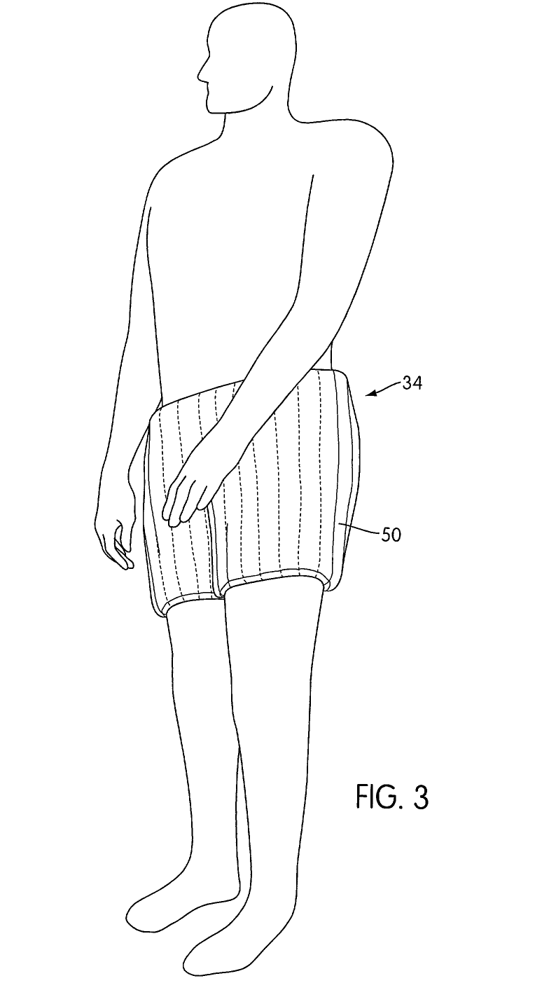

The pad can also be formed into other types of garments for treating other body areas. For example, the garment could be formed as a leg sleeve 38 as shown in FIG. 2, or as a brief 34 as shown in FIG. 3 for treating the pelvic area, or a widened belt (not shown) for treating the abdominal area, or a vest (not shown) for treating the upper chest and/or back.

The foam pad is encased between the inner and outer layers 24, 26 which are secured to one another, preferably by sewing. A plurality of channels 36 are formed in the pad. The channels 36 are spaced apart from one another and are preferably oriented in a direction corresponding to a longitudinal (desired fluid flow) direction of the limb or other body area being treated. For example, in the sleeve shown in FIG. 1, the channels 36 (which are on the inner surface of pad 14 and not visible in FIG. 1) extend from the distal portion of the limb (i.e., the hand and wrist area) to the proximal portion of the limb (i.e., the shoulder and armpit area). The channels 36 are formed by bringing the inner and outer layers 24, 26 into close proximity to one another, preferably in areas at or near the valleys 32 disposed between adjacent protrusions 30 of the foam pad 22. In a preferred embodiment, the channels 36 are stitched seams wherein the inner and outer layers 24, 26 are sewn to one another through generally longitudinally-extending valleys of the foam pad 22 with the base portion of the foam pad sandwiched between the inner and outer layers along the stitched seam.

When in use on a body area, the protrusions 30 of the foam pad 22 produce a surface pressure on the body area that is greater than the pressure formed by the intermediate valleys 32 as well as the pressure formed by the longitudinally-extending channels 36. When worn by a patient, the apparatus 10 promotes venous lymphatic aspiration and redistribution of the edema. In particular, the discrete pressure points provided by the protrusions 30 stimulates fluid redistribution while the low pressure areas of the valleys 32 permit and facilitate transverse flow of the fluid into the channel regions 36, and the channel regions 36 facilitate longitudinal flow of the fluid from the distal area of the limb to the proximal area of the limb.

In a preferred embodiment, a securing mechanism 50 (such as a zipper or Velcro strips 46, 48, and preferably Velcro strips) is provided along the opposed edges 16, 18 of the pad 14. The securing mechanism 50 can be closed to form the sleeve (or other garment) prior to the patient pulling the garment onto the body area to be treated. The securing mechanism 50 (i.e., Velcro) permits the pad to be unrolled and easily removed from the body area so that the patient can take the garment off with little or no assistance.

As shown in FIG. 4 in a preferred embodiment for an arm sleeve, a distal end of the pad is sewn together as shown at 40 with a hole provided (not shown) for the thumb and an end hole provided at 44 (see FIG. 1) for the fingers and hand.

While the invention has been described in connection with what are presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

What we claim is:

1. An apparatus for treating an elevated concentration of interstitial fluid in a body area of a patient including a pad constructed and arranged to be placed in pressure-applying, conforming relation to the body area, said pad comprising:
an outer liner;
an inner liner connected at marginal edges thereof to said inner liner;
a multiplicity of resilient protrusions encased between said inner and outer liners; and
a plurality of channels formed in areas between said protrusions by securing said inner liner in close proximity to said outer liner along each said channel,
wherein said channels and said protrusions are constructed and arranged such that when said pad is placed in pressure-applying, conforming relation to the body area, localized pressure generated on the body area at each said protrusion is greater than localized pressure generated on the body area along said channel.

2. The apparatus of claim 1, wherein said pad is formed into a sleeve to be worn on a limb of the patient so as to provide pressure-applying, conforming contact of said pad completely surrounding the limb.

3. The apparatus of claim 1, further comprising a securing device along cooperating edges of said pad to secure said cooperating edges to each other to secure said pad to the body area.

4. The apparatus of claim 3, wherein said securing device comprises cooperating Velcro strips attached to each of said cooperating edges.

5. The apparatus of claim 1, wherein said inner and outer layers comprise cloth.

6. The apparatus of claim 4, said cloth comprising substantially cotton.

7. The apparatus of claim 1, wherein said pad further comprises a foam pad encased between said inner and outer liners, said foam pad comprising a base portion with said protrusions protruding therefrom.

8. The apparatus of claim 1, wherein each of said channels comprises a seam sewn between said inner and outer layers.

9. The apparatus of claim 1, wherein said channels are arranged to extend along said pad from an edge of said pad corresponding to a distal end of the body area to an edge of said pad corresponding to a proximal edge of the body area.
